(19) United States
(12) Patent Application Publication
(10) Pub. No.: US 2014/0357519 A1
(43) Pub. Date: Dec. 4, 2014

(54) METHODS OF SCREENING MONOCLONAL ANTIBODY POPULATIONS

(71) Applicant: Baylor College of Medicine, Houston, TX (US)

(72) Inventors: Michael A. Mancini, Houston, TX (US); Dean P. Edwards, Bellaire, TX (US)

(73) Assignee: BAYLOR COLLEGE OF MEDICINE, Houston, TX (US)

(21) Appl. No.: 14/372,570

(22) PCT Filed: Jan. 16, 2013
(86) PCT No.: PCT/US13/21665

§ 371 Date: Jul. 16, 2014

Related U.S. Application Data
(60) Provisional application No. 61/587,111, filed on Jan. 16, 2012.

(51) Int. Cl. G01N 33/577

(52) U.S. Cl. CPC G01N 33/577; G01N 2333/46; G01N 2223/612; G01N 2458/00
USPC 506/9

(57) ABSTRACT

The present invention concerns methods and compositions for screening primary hybridoma cultures to generate monoclonal antibodies that are useful in a variety of methods, including for in situ cellular imaging by immunocytochemical assays or in vivo applications, for example. Embodiments of the methods concern the use of automated high-throughput immunofluorescence (for example) to identify subcellular in vivo patterns that are expected based on the antigen or that may be unforeseen.

This application claims priority to U.S. Provisional Patent Application Ser. No. 61/587,111, filed Jan. 16, 2013.

### STATEMENT REGARDING FEDERALLY SPONSORED RESEARCH OR DEVELOPMENT

This invention was made with government support under P30CA125123 awarded by NIH/NCI; RC2ES018789, awarded by NIH/NIEHS; RO1 CA46938, awarded by NIH/NCI; P01 GM081627 awarded by NIH/GM; and DK049030 awarded by NIDDK. The government has certain rights in the invention.

### TECHNICAL FIELD

The present invention concerns at least the fields of immunology, cellular biology, and molecular biology.

### BACKGROUND OF THE INVENTION

Standard production of monoclonal antibodies employs the use of hybridomas. In particular, following immunization of mice with an antigen of interest, immune cells are isolated and fused with myeloma cells, and the resultant hybridomas are screened by ELISA, clonally expanded, and verified, for example by Western. Often in the art, antibodies screened in this manner fail to be useful beyond the assay used during screening (such as ELISA or immunoblot) and, for example, fail to work in immunofluorescence, immunohistochemistry, immunoprecipitation, reverse phase protein arrays, and/or chromatin immunoprecipitation. The present invention provides a long-felt need in the art to utilize a high throughput microscopy-based screening of monoclonal antibodies that are applicable for use in a variety of methods.

### BRIEF SUMMARY OF THE INVENTION

The present invention is directed to methods and compositions that concern screening for monoclonal antibodies of interest. In specific embodiments, the methods of the invention concern the raising of monoclonal antibodies using high throughput microscopy (HTM)-based hybridoma screening, and in certain embodiments the HTM-based hybridoma screening employs in vivo subcellular localization of one or more targets. In aspects of the invention, high throughput immunofluorescence is used to screen monoclonal antibodies.

In some embodiments, the present invention encompasses automation of monoclonal antibody screening using high content analysis.

In some embodiments, the present invention includes parallel usage of automated fluorescence microscopy during primary and follow-on screening. In specific embodiments, the methods generate “on-target” hits that include monoclonal antibodies that recognize an expected and known protein of interest, whereas in some embodiments the methods alternatively or additionally generate “off-target” hits that include monoclonal antibodies that recognize an unintentional protein and/or recognize one or more proteins or subcellular components that were not expected.

In some embodiments of the invention, the antigen employed for screening of the monoclonal antibodies are present in a cell, and in at least certain aspects the antigen is labeled. In embodiments wherein more than one antigen is employed for screening of the monoclonal antibodies in vivo, different antigens are differentially labeled (for example, with different colored labels), introduced to cells either individually or in combination, with individual or multi-labeled cells being screened simultaneously in parallel or mixed cell cultures.

In particular embodiments, the monoclonal antibodies are screened against a mixture of cells. The mixture may include cells that harbor the labeled antigen(s) and also cells that are negative controls; negative control cells may be cells that lack the antigen or express mutant antigen(s). Screening against such a mixture allows screening to define antigen-specific hits. In some cases, the mixture of cells are exposed to environmental conditions that initiate particular subcellular localization of the antigen, such as nuclear translocation, plasma membrane localization, or targeting to any subcellular organelle/structure, trafficking between organelles, translocation to the plasma membrane, localization to leading cell edge or membrane ruffles, trafficking through endocytic vesicles, etc.

In some embodiments of the invention, the methods to screen monoclonal antibodies is automated. An exemplary workflow of the analysis of the monoclonal antibodies may include analysis of the hybridoma cultures on multi-well plates, consideration of reference controls, and high throughput microscopy resulting in image analysis. Such a method may include one or more of the following: 1) pre-processing of the visuals to correct images to remove background fluorescence and apply flat-field correction from the multi-well plates, 2) segmentation of the field into single cell regions using cell reference channels (i.e., DAPI/nuclei), 3) measurement of the images that includes extracting features from image channels (e.g., nucleus:cytoplasm ratio), 4) filtering that removes single cells samples with abnormal measurements, 5) identification of the specific antibodies of interest that includes correlating expression of the labeled antigen with antibody intensity in the particular subcellular localization, and, in at least some cases, identification of specific off-target hits by training a system to automatically recognize subcellular patterns in a reference dataset.

In some embodiments of the invention, in screening the hybridomas one can utilize reference antibodies to identify organelle labeling patterns, for example, and the automated system can be trained to recognize and classify particular patterns and, in at least some cases, apply the information to multiple primary hybridoma wells. Exemplary patterns include those in which the antibodies target the nucleus, cytoskeleton, Golgi apparatus, mitochondria, nucleoli, membrane, cytoplasm, intermediate filament, endosomes, lamina, nucleoplasm, and so forth.

Following automated microscopy and image analysis resulting in determination of one or more antibodies of interest, such “hits” can be confirmed by mass spectrometry and/or immunoblotting, for example.

The antigen(s) initially immunized in the mouse to produce the primary hybridoma cultures may be of any kind. In specific embodiments, the immunization includes proteins, subcellular structures, subproteomes (e.g. membrane proteins, exosomes), organelles (e.g., nuclei, nucleolus, ribosome, vesicle, rough endoplasmic reticulum, Golgi apparatus, cytoskeleton, smooth endoplasmic reticulum, mitochondria, vacuole, cytosol, lysosome, and/or centriole, microorganisms), cellular extracts, including from differentiated cells, undifferentiated cells, stem cells, tumor cells, drug- or hormone-resistant tumor cells, hormone or growth factor sensitive and hormone or growth factor resistant cells, normal vs. cancer cells, cancer cells from different stages of progression, normal cells from different stages of development, and the extract may be a nuclear extract, membrane extract or protein or protein/DNA complexes, and so forth chromatin fractions, isolated organelles, subcellular fractions, etc.

In some embodiments, there is a method of using high throughput microscopy to screen primary hybridomas following the production of monoclonal antibodies, comprising the step of testing for the subcellular co-localization of a labeled antigen of interest or one or more differentially labeled antigens of interest with one or more monoclonal antibodies and also testing for a negative result against a negative control. In certain embodiments the co-localization is compared to a subcellular pattern of interest. In specific embodiments, the method includes exposing a plurality of hybridoma cell cultures to a mixture of cells that includes cells producing the labeled protein of interest and cells that lack the protein of interest. In certain aspects of one or more methods of the invention, there is assayed co-localization of a test monoclonal antibody with a particular localization pattern for a subcellular structure and/or for co-localization with a pattern of one or more particular reference antibodies.

In some embodiments, there is a method of screening primary hybridoma cultures for monoclonal antibodies generated in response to a collection of antigens, including one or more proteins or subcellular structures or organelles, protein complexes, or subcellular extracts, wherein the method uses high throughput immunofluorescence microscopy, the method comprising the steps of: generating a plurality (including thousands, for example) of hybridoma cell cultures and separating (for example separating them by single cell cloning and growth as colonies derived from a single cells) at least a plurality of the cultures (for example, in multi-well plates or by roboticized picking of clones); providing to cells that are harboring the labeled antigen(s) of interest antibodies from at least one of the cultures; and assaying the subcellular localization of the labeled antigen:antibody complex including assaying for absence of signal in negative control cells lacking the antigen(s) or expressing mutant protein(s). The skilled artisan recognizes that in any of the methods of the invention multiple antigens being assayed in the same method would require differential labeling such that the different antigens could be distinguished.

In some embodiments, there is a method of screening hybridoma cultures for one or more antibodies of interest, comprising the steps of obtaining or producing hybridoma cultures produced from mice immunized with one or more purified or partially purified antigens of interest, or antigen(s) from a subcellular structure/fraction, said cultures producing a variety of test monoclonal antibodies; providing a mixture of cells, wherein some cells in the mixture have a known labeled antigen(s) of interest and some cells in the mixture lack the antigen(s) of interest; exposing the mixture of cells to test antibodies from a selected hybridoma culture to produce a complex of test antibody/antigen of interest; exposing the complex to a labeled secondary antibody (anti-mouse IgG or other isotypes as desired) that binds to the test monoclonal mouse antibody; and determining the subcellular localization of the protein of interest by detecting the label. In particular embodiments, the method further comprises the step of using high throughput microscopy to simultaneously determine the subcellular localization of multiple antigens of interest. In specific embodiments, test antibodies that do not bind the antigen of interest are identified by subcellular localization that is non-identical to the localization pattern of the antigen of interest and in at least certain aspects are identified upon comparison to a reference pattern.

In some cases there are embodiments that employ the high throughput screening methods of the invention to compare antibody hybridoma cultures between two or more different types of cells or between the same type of cell but exposed to a cell-specific stimuli, for example. In some embodiments, antibody hybridoma cultures from differentiated cells vs. non-differentiated cells are compared. In other cases, antibody hybridoma cultures from tumor cells vs. normal cells (or drug-sensitive vs. drug-resistant cancer cells or other cell types, such as hormone or growth factor sensitive vs. resistant cells) are compared.

In some embodiments, there is a method of screening primary hybridoma cultures for one or more antibodies of interest, comprising the steps of: providing a plurality of primary hybridoma cultures generated from immunization of a non-human animal with one or more antigens; performing a first screen to determine specificity of a test monoclonal antibody for the antigen; performing a second screen of the test monoclonal antibody by exposing the test monoclonal antibody to a (in specific embodiments, substantially equal) mixture of cells in which some cells in the mixture have the antigen in labeled form and some cells in the mixture lack the antigen (in specific embodiments, the exact ratio is not important, but in cases wherein statistical analysis is employed, substantially equal numbers is useful); where possible, this is done in the first screen; assaying for in vivo co-localization of the test monoclonal antibody with the antigen; and assaying for the absence of signal from the label in the cells that lack the antigen.

In specific embodiments, the assaying for in vivo co-localization of the test monoclonal antibody with the antigen is further defined as assaying for binding of a labeled second antibody to the test antibody. In specific embodiments, the antigen is a protein, a protein fragment, peptide, cellular extract, an organelle, subcellular structure, subproteome, or mixture thereof. In particular aspects, one or more of the steps are performed concomitantly. In certain aspects, the first screen comprises ELISA, western, or a combination thereof. In at least some cases, the label is fluorescent, colored, radioactive, or a combination thereof. In particular embodiments, the antigen in labeled form is further defined as being a fusion protein that comprises a protein region that is detectable by color or fluorescence. In specific embodiments, the test monoclonal antibody is measured by the intensity of the label of the secondary antibody. In specific aspects, the co-localization has a known co-localization pattern, for example one that is indicative of a subcellular structure, such as an organelle, including an organelle selected from the group consisting of nuclei, nucleolus, ribosome, vesicle, rough endoplasmic reticulum, Golgi apparatus, cytoskeleton, smooth endoplasmic reticulum, mitochondria, vacuole, cytosol, lysosome, and/or centriole.

In specific embodiments, the co-localization has a known pattern and wherein the method further comprises the step of assaying for antibodies that localize with a subcellular in vivo pattern that is not identical to the known co-localization pattern. In at least some methods of the invention, the method is automated. In certain aspects, test antibodies from more than one primary hybridoma culture are screened concomitantly. In specific embodiments, the in vivo co-localization is assayed subsequent to treatment of the cells that have the antigen with a cellular signal that results in subcellular movement of the antigen.

In some embodiments of the invention, there is a method of screening primary hybridoma cultures for one or more antibodies of interest, comprising the steps of: providing a plurality of primary hybridoma cultures generated from immunization of a non-human animal with one or more antigens; assaying (for example, by secondary immunofluorescence, such as by fluoro-conjugated secondary antibodies or any visibly-detectable conjugate (e.g., nanoparticles, or light diffracting conjugates or beads)) for in vivo localization of monoclonal antibodies from one or more primary hybridoma cultures; and comparing the localization pattern of the antibodies to the pattern of one or more known cellular features.

In some embodiments, there is a method of producing a plurality of antibodies that recognize a subproteome, comprising the steps of: providing a plurality of primary hybridoma cultures generated from immunization of a non-human animal with a subproteome; assaying for in vivo subcellular localization of antibodies from one or more cultures, thereby producing a pattern recognition for the antibodies; and comparing the pattern to a cellular pattern for one or more known or unknown epitopes or components of the subproteome. In specific embodiments, the method further comprises using antibodies that recognize the known epitope or component of the subproteome to bind to its respective antigen among a variety of proteins. In certain embodiments the method is further defined as using the antibodies to recognize the known epitope or component of the subproteome in mass spectrometry, gel electrophoresis, immunoblotting, or a combination thereof. In specific aspects, the subproteome is a purified or partially purified protein complex, such as a transcriptional regulatory protein complex.

In some embodiments, there is a method of screening primary hybridoma cultures for differences in antibody in vivo subcellular localization between two or more cell populations, comprising the steps of: providing a plurality of primary hybridoma cultures generated from immunization of a non-human animal with a first cell population; providing a plurality of primary hybridoma cultures generated from immunization of a non-human animal with a second cell population; in at least some cases optionally exposing the test monoclonal antibody to a mixture of cells in which some cells in the mixture have the antigen in labeled form and some cells in the mixture lack the antigen; assaying for in vivo subcellular localization of one or more cultures from the respective cell populations. In specific embodiments, the first and second cell populations are further defined as: a) differentiated vs. undifferentiated cells; b) cancer vs. non-cancer cells; c) cancer drug-resistant vs. cancer drug-sensitive cells; d) hormone or growth factor sensitive and resistant cells; e) cells from different stages of cancer progression; or f) cells of different tissue types.

Methods of the invention can be useful for cell culture and biochemical research and diagnostic testing, and including in vivo applications. In specific aspects, the obtained antibodies are useful in vivo. This is particularly true if one specifically screens for hybridomas that have neutralizing functions based upon an in vitro primary screen (for example, looking for neutralizing membrane fraction antibodies that, through inhibition of cell surface receptors, suppress constitutive activity of a transcription factor, such as one related to cancer, for example prostate cancer).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is an illustration of raising immunofluoresence (IF) qualified monoclonal antibodies to the unique N-terminal domain (NTD) of the B isoform of human progesterone receptor (PR-B) using high throughput microscopy based hydridoma screening. DBD (DNA binding domain) of PR, LBD (ligand binding domain), aa sequence numbering of PR (aa 1-933). Antigen was purified NTD of PR-B (aa 1-164);

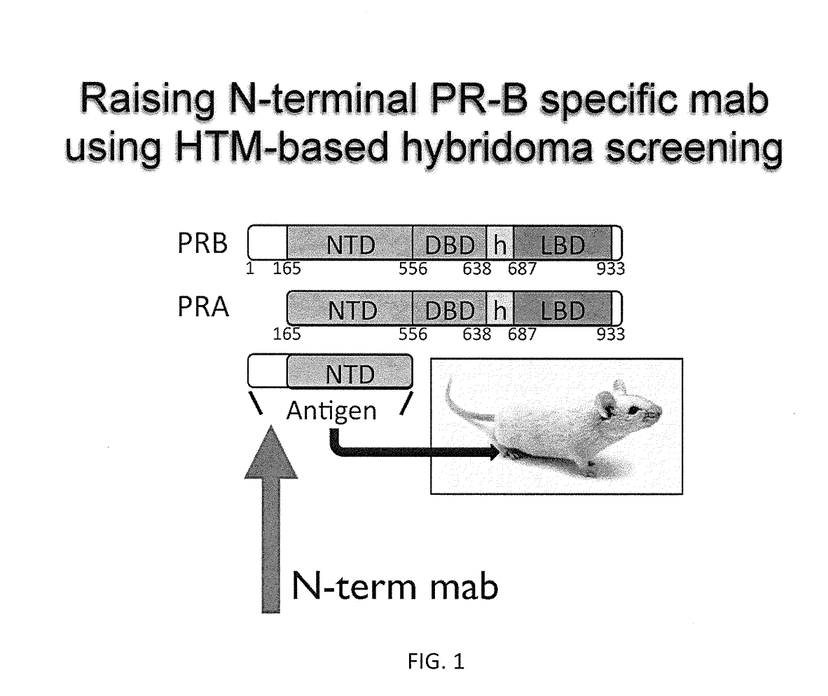

FIG. 2 is an illustration of GFP-tagged antigen (PR-B) positive and negative cells used for primary high throughput immunofluoresence (IF) monoclonal antibody screening;

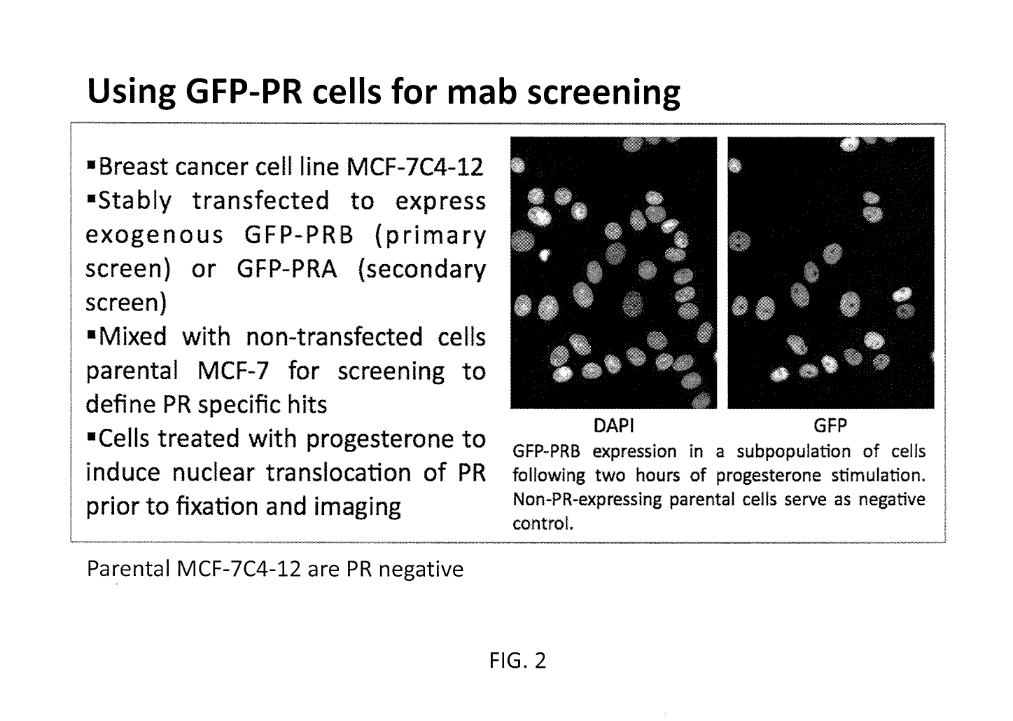

FIG. 3 is an exemplary workflow embodiment of the invention for high throughput microscopy based primary screening of hydridomas;

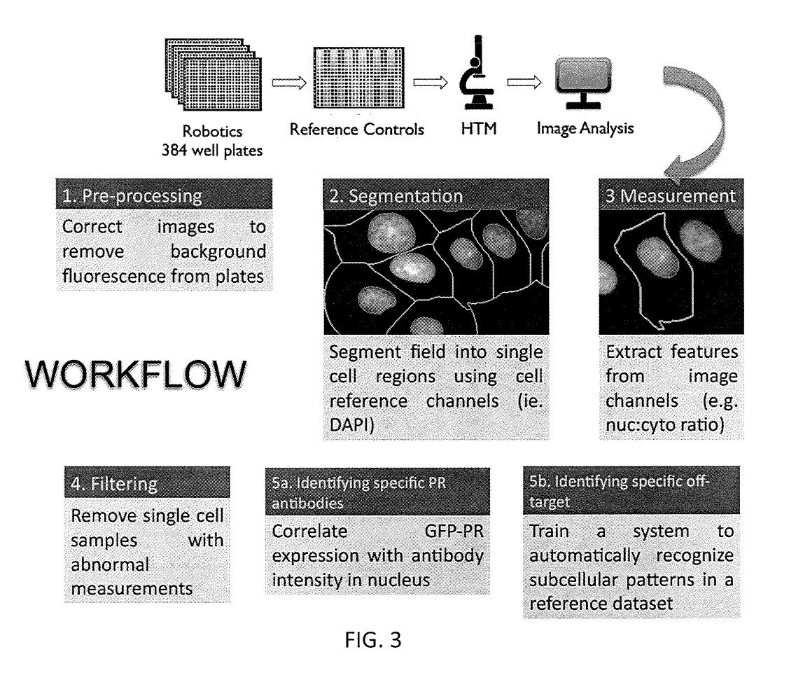

FIG. 4 demonstrates how the primary high throughput microscopy screen of hybridomas identified IF qualified Mabs for specific detection of the B isoform of PR by co-localization of nuclear IF signals with nuclear GFP-PR-B positive cells;

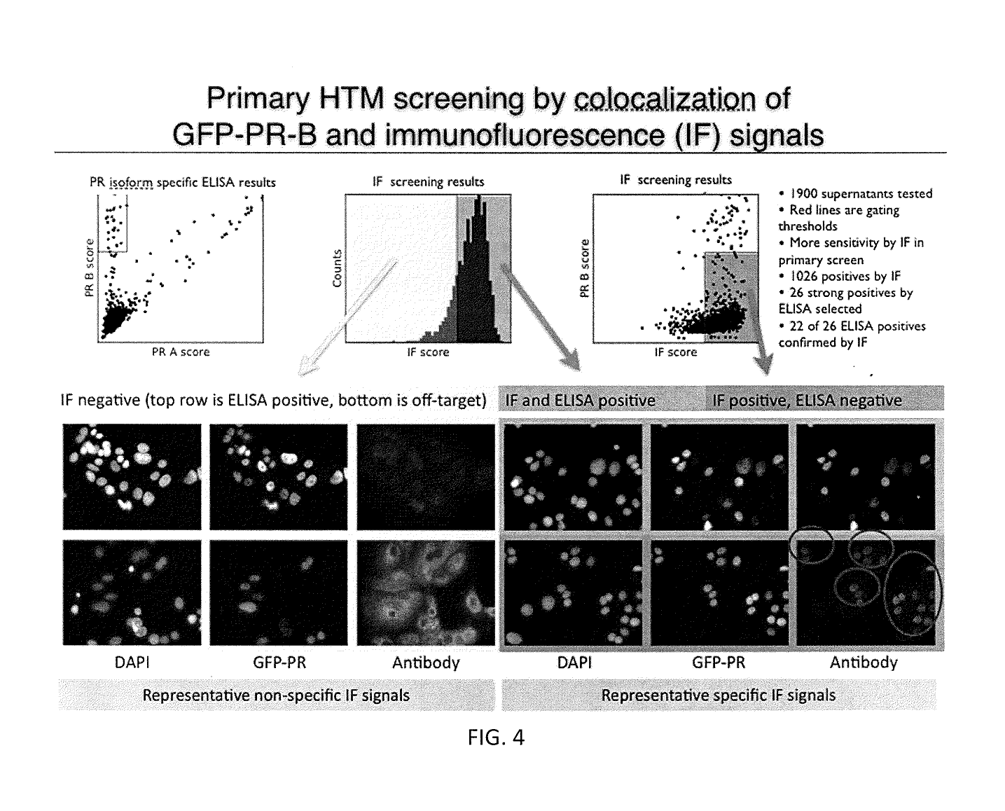

FIG. 5 illustrates an exemplary embodiment of the secondary HTM IF screening with GFP-PRB vs. GFP-PRA expressing cells to select for MAbs specific for PR-B;

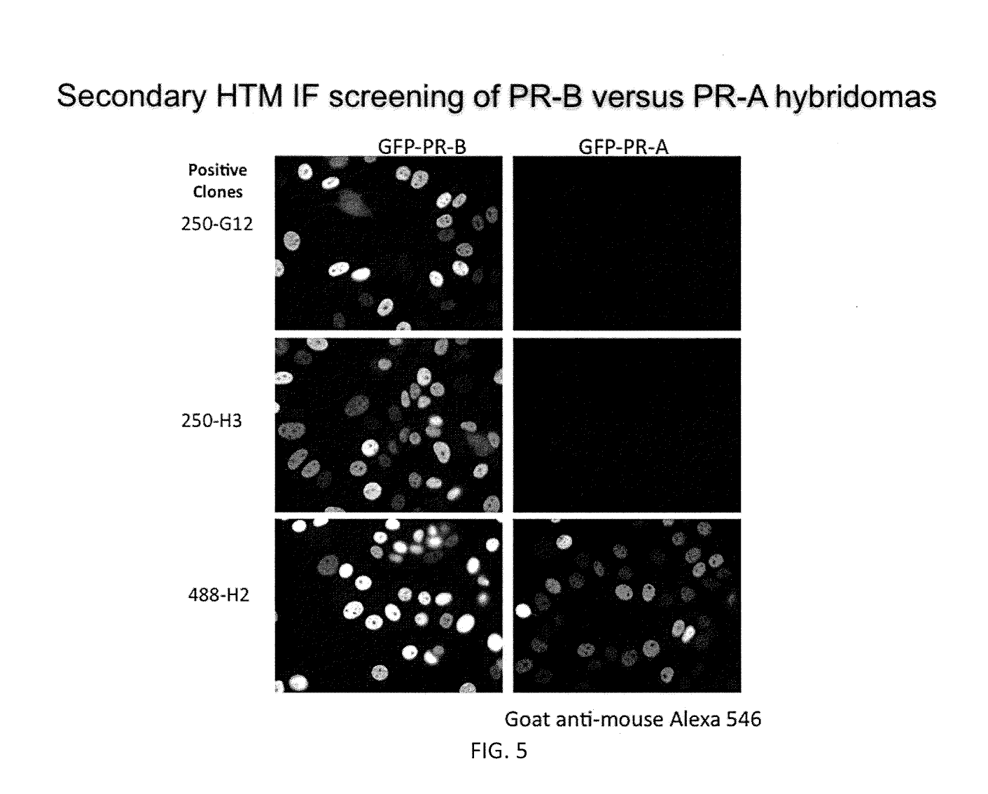

FIG. 6. Western immunoblot assay confirming the specifity of the MAbs for either the B-isoform of PR or for both PR-A and PR-B;

FIG. 7. Provides exemplary workflow for HTM identification of off-target antigens;

FIG. 8. Provides examples of subcellular patterns of IF staining identified by additional off-target MAbs;

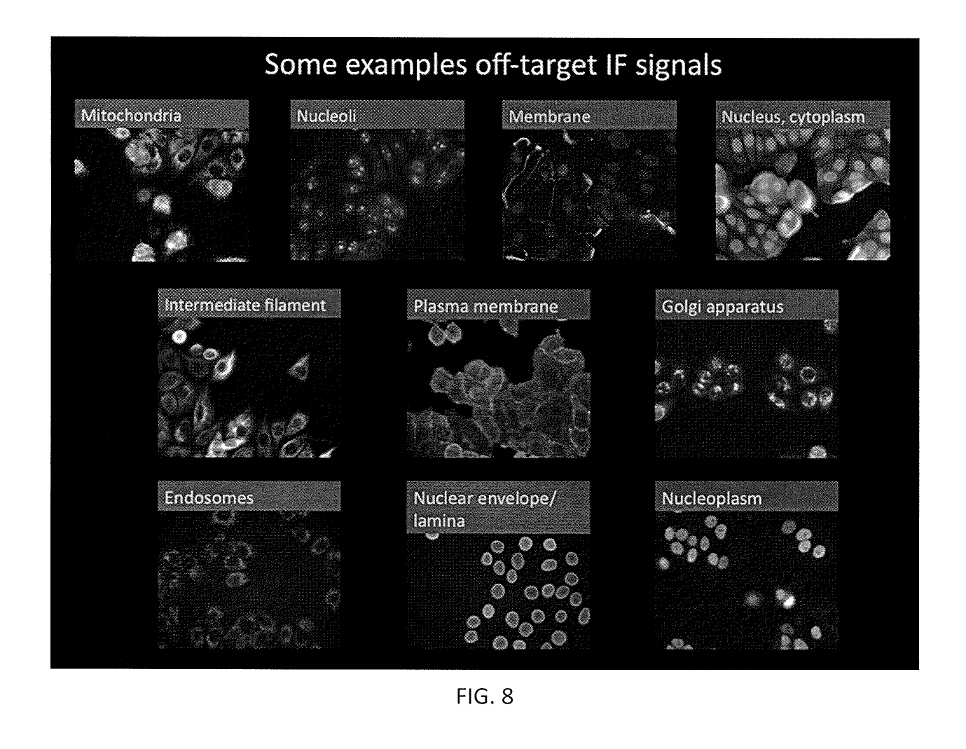

FIG. 9 illustrates an embodiment of the method wherein immunofluorescence qualified monoclonal antibodies are identified by a work flow of primary IF screening of hybridomas from mice injected with purified protein antigen of interest or subproteomes to identify multiplexed proteins or subproteomes (“reverse proteomics”).

### DETAILED DESCRIPTION OF THE INVENTION

### I. Exemplary Definitions

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “antigen” as used herein refers to one or more entities utilized to immunize an animal for the generation of monoclonal antibodies thereto. The antigen may include a protein or part of a protein, or other cellular molecule, subcellular structure, subproteome, organelle, whole cell or subcellular lysates, synthetic peptide or other small molecules conjugated to a hapten, whole microorganisms, and so forth.

The term “subproteome” as used herein refers to a purified or partially-purified subcellular fraction(s), multi-protein complex or protein machinery, isolated organelle, isolated cell membranes, isolated chromatin fractions, etc.

### II. Methods of Screening for Specificity of Monoclonal Antibody Libraries by High Throughput Microscopy

In some embodiments, the inventors have developed a new workflow that allows rapid multiparametric-based identification of antibody specificity and sensitivity during the initial and all subsequent stages of screening during monoclonal antibody (MAb) production. In certain embodiments, the workflow utilizes a combination of 1) multi-channel high throughput immunofluorescence microscopy, 2) engineered cell lines with internal fluorescent- (or epitope) tagged or immunofluorescently-labeled reference proteins, or cytological dyes, and 3) custom image analysis tools to perform rapid and accurate pattern recognition and correlation analyses. The workflow assures that selection of primary hybridomas slated for expansion and subsequent single cell cloning will be those producing the highest quality MAbs (to antigen or subcellular structure) in terms of specificity and sensitivity for the most rigorous applications such as immunocytochemistry and other assays that require detection of antigen in intact cells/tissues or total cellular protein extracts (e.g., reverse phase protein arrays or immunoprecipitation).

This new process is markedly different from the standard MAb production workflow that typically employs an enzyme-linked immuno-absorbent assay (ELISA) with the target antigen immobilized to a microtiter plate (for example) as a key assay to drive selection during the first rounds of screening. Primary screening is typically followed by Western immunoblotting to determine specificity for a single protein band of the appropriate molecular mass. Whether MAbs selected by the traditional workflow are of sufficient quality and specificity for cell- or tissue-based immunolabeling assays (e.g., immunofluorescence, immunocytochemistry, immunohistochemistry) and other whole or partial cell lysate-based assays (reverse phase protein arrays and other antibody array platforms) is left to chance and, very frequently, they are not. In embodiments of the invention, there is enhancement of the efficiency of the entire hybridoma process in selecting the highest quality, most versatile MAbs for these more demanding applications.

An exemplary illustration of the one particular embodiment has been performed with production, screening and validation of MAbs to the B isoform of human progesterone receptor (PR). PR-B has a unique extension of the amino terminal domain (NTD), while PR-A has a shorter truncated NTD that is otherwise identical to PR-B. An NTD containing both the unique N-terminus of PR-B and the shared region with PR-A was expressed as a recombinant protein, purified and used to immunize Balb/c mice. Splenocytes from an immunized mouse were fused with immortalized mouse myeloma cells by standard MAb procedures and ˜1,900 hybridoma cell cultures were generated in 20×96 well microtiter plates. Standard ELISA against purified NTD of PR-B vs PR-A, as well as full-length PR, was performed as the primary and secondary screen, with Western immunoblot as the tertiary screen for mono-specificity of the MAbs for a single PR protein band. In parallel, primary and secondary screenings were also performed by high throughput immunofluorescence microscopy (HTM) with breast cancer cell lines engineered to stably express PR-B, or PR-A, as a GFP (green fluorescent protein) fusion protein which were mixed with non-PR expressing parental cells (˜50% each). Using an image analysis and data mining approach, image data from all wells (18 images/well×1900) were rapidly (˜12 hrs) examined to define whether hybridomas produced antibodies that specifically and precisely colocalized with nuclear GFP-PR-B. PR-B in the absence of progesterone is distributed between the cytoplasm and nucleus. Cells for screening were treated for 1 hr with the synthetic progestin R5020 to translocate all PR-B to the nucleus. As negative control, parental cells lacking PR were available in the same well (e.g., non-green nuclei); thus, specificity of the MAbs for PR was assured from the initial screening. Further, the sensitivity of the antibody was also examined at the same time, reflected by the pixel intensity of the nuclear MAb signal. Lastly, as the breast cancer cells were pre-fixed with a formaldehyde treatment akin to the type of protocols used in diagnostic pathology assays (immunohistochemistry—IHC of tissue sections), the initial screen also selects for MAbs most likely to work by IHC, and thus ensures their versatility for multiple assays, in certain aspects as antibodies that recognize their antigens in fixed cells/tissues routinely work in biochemical assays. This is particularly important, as a key problem with selection of MAbs based on ELISA and Western immunoblot assays is that they frequently fail to work in other assays like immunofluorescence or IHC.

Subsequently, positives cultures were assessed for specificity for the unique NTD of PR-B by directly comparing immunofluorescence nuclear co-labeling with cells engineered to express fusions of green fluorescent protein (GFP) with PR-B or PR-A (e.g., PR-B-GFP or PR-A-GFP). The IF labeling results for specificity for NTD of PR-B were confirmed by ELISA and Western immunoblots of recombinant PR protein domains and full length PR-A and PR-B

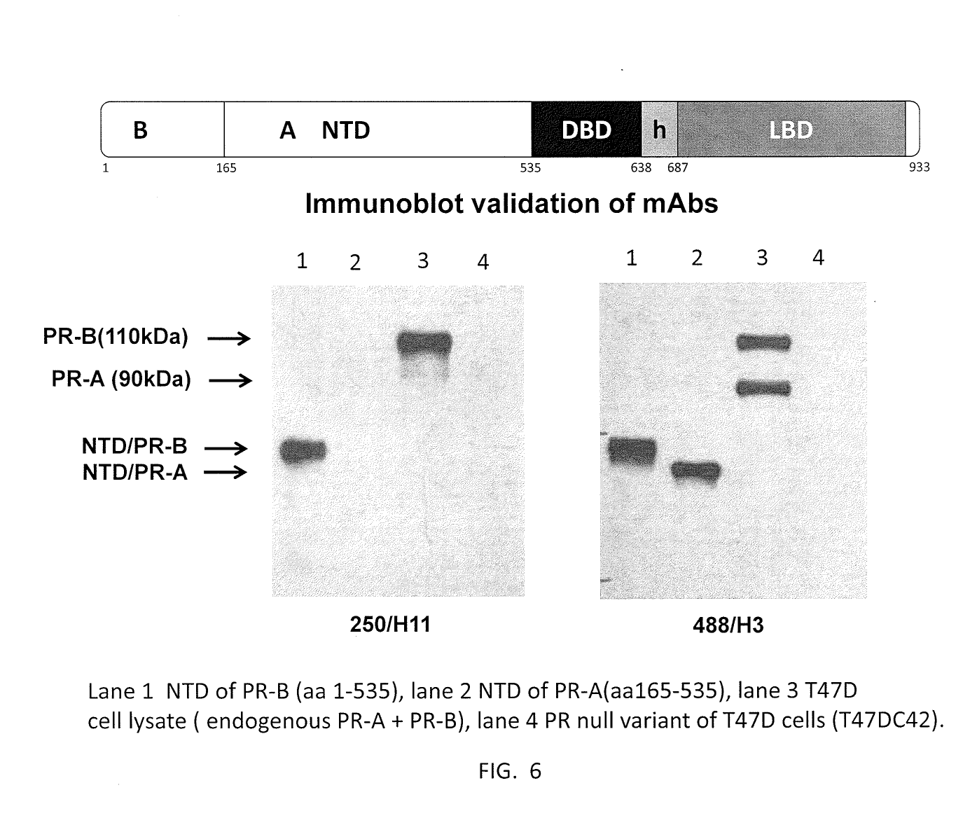

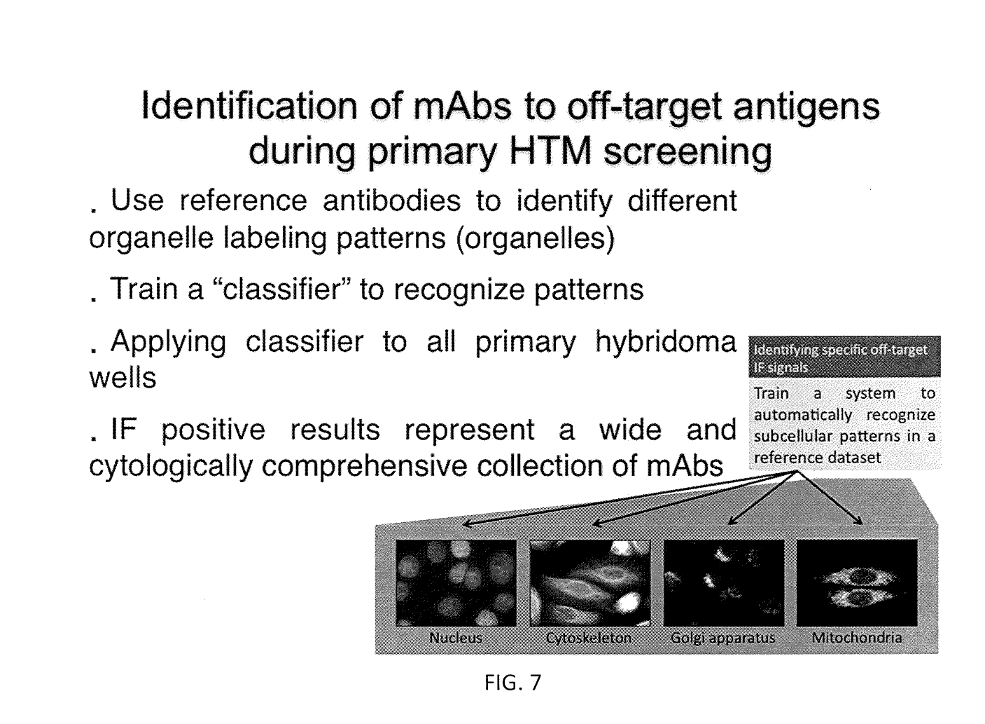

Ultimately, specific embodiments of an exemplary workflow identified and qualified a large number of PR-B specific monoclonal antibodies (>1000 hybridomas in the primary HTF IF screening were positive, whereas 10-fold fewer were positive by ELISA; 12 mAbs were selected as the highest quality for final isolation/characterization) against the unique NTD of PR-B and others that are against the common region of the NTD that cross react with both PR-A and PR-B. All the final selected MAbs were highly specific and sensitive for detection of PR by immunofluorescence (IF) and were confirmed by Western immunoblotting to be monospecific for the correct PR isoform (FIG. 6). The cross referencing specificity and sensitivity between IF and biochemical approaches ensured that only the highest quality MAb clones were selected and expanded. Subsequently, most of the MAbs were also confirmed to be highly specific and sensitive for detection of PR-B in formalin fixed paraffin embedded sections of different breast cancer derived tumors (FIG. 7). The exemplary workflow also identified some PR MAbs there were positive by IF and subsequently confirmed by Western immunoblotting that were not detected by the primary ELISA screen and thus would have been totally missed by the traditional workflow. Thus the primary HTM screening is more sensitive than traditional biochemical hybridoma screening methods, in at least some embodiments.

In embodiments of the invention, one feature of this process is the ability in the initial screen of hybridomas to determine MAb specificity by IF assay by HTM analysis of a mixture of fluorescent (GFP, RFP, or other visible epitopes, etc.) protein of interest (POI) tagged positive and negative cells. Because IF assay signals are generated in the natural environment of the cell, cross reaction with epitopes other than the POI cannot be tolerated and demands a much more rigorous specificity than Western immunoblots that can separate and identify cross reactive proteins by size using gel electrophoresis. In the traditional MAb workflow, specificity of MAbs for IF is evaluated only at the end of the ELISA/immunoblot process. In some cases, the ELISA/immunoblot MAbs can be tested by performing IF in response to an activation signal and/or RNAi knock down of the POI; essentially, one then hopes for the best as the selection process is complete (e.g., no further clones are available for testing from the immunized animal). This approach often fails to generate MAbs with the required specificity and sensitivity to work under both biochemical and IF conditions. In embodiments of the invention, there is the equivalent of testing the effect of knock down of the POI on IF assays at all steps of the screening process with final Western immunoblotting or other biochemical assays to be used to cross-confirm specificity of the MAbs.

This exemplary workflow is amenable to any MAb project that can colocalize the POI (or AOI, antigen of interest, when immunogenes may include carbohydrate or other non-protein antigens) by immunofluorescence with an internally fluorescence reference protein (e.g., GFP), or other epitope tag (e.g., Flag, HA, etc.), or known high quality reference antibody. This process should also be applicable for multiplex immunization of up to 3 (for example, or more) different proteins of interest using a multi-colored approach with reference proteins tagged with different fluorescent probes or epitopes, to simultaneously screen MAb specificity for each antigen. In specific embodiments, this approach is employed for immunizations with multi-protein complexes, subproteomes (e.g., subsets of total cellular proteins) or organelles (e.g., Golgi, nuclei, etc.) as immunogens by colocalization of IF signals with any fluorescently labeled cytological structure. For example, if a mitochondrial protein was the protein of interest, red fluorescent mitochondrial dyes (or reference antibody to a known mitochondrial protein) could be used as a reference marker for the image analysis to define precise colocalization with a potential new MAb. The speed, sensitivity and specificity of embodiments of the method marks a substantial advancement in generation of high quality monoclonal antibodies.

### III. Novel Multiplex Production of Monoclonal Antibodies to Subproteomes by Use of Automated High Throughput Immunofluorescent Pattern Recognition

The production and use of monoclonal antibodies (mAbs) is inseparable to an enormous variety of biological uses, particularly in the context of proteomics. High quality mabs are used in a variety of immuno-techniques to define protein molecular weight, characterization of protein-protein interactions, gene regulator and chromatin-associated complexes and microscopy-based approaches to determine tissue, cellular and subcellular location, and immunohistochemistry for analysis of markers of disease in clinical pathology laboratories, for example. The long-standing approach to generate monoclonal antibodies includes immunization of mice (or other species, such as rat, hamster, guinea pig, or rabbit) with purified protein or representative synthetic peptide (for example), then harvesting antibody-producing cells from the spleen and immortalization by cell fusion with non-immunoglobulin-secreting myeloma tumor cells; typically, up to around 2,000-4,000 wells of a multi-well culture dish (for example) are plated to start the screening for useful monoclonal antibody-producing hybridoma clones.

The general process of selection of viable monoclonal antibody-producing hybridoma clones is based upon defining which of the primary cultures are producing immunoglobulin, for example by enzyme-linked immunoabsorbant assay (ELISA), and whether or not there is an antibody that is specific to the immunized protein. Speedy ELISA results are essential to determine which wells are to be expanded and subcloned; typically this can involve many dozens of hybridoma cultures that are retested by ELISA and/or immunoblot, and then single cell cloned. Lastly, cloned cell lines are expanded and retested, with the end result being production of large quantities of a specific monoclonal antibody.

Because of the nature of the screening process, monoclonal antibodies that are selected will work in biochemical assays; for a wider range of uses, such as in immunopreciptiation of immunofluorescence, the monoclonal antibody has to be tested anew. Unfortunately, it is not uncommon that monoclonal antibodies that are selected based upon biochemical testing (ELISA/immunoblot) fail to work in other assays.

In order to improve the ability to generate specific monoclonal antibodies that are compatible with immunofluorescence, for example, the inventors have used roboticized microfluidics, automated fluorescence microscopy and customized automated image analysis software to greatly increase the speed and accuracy of selecting monoclonal antibodies for expansion from the original primary hybridomas. As immunofluorescence is used throughout the entire process, by definition the selected, expanded and clonal monoclonal antibodies function for subcellular localization of the recognized epitopes. Because the initial screening and interpretation of primary hybridomas is a time-critical process, requiring a ‘go-or-no-go’ answer for all clones within 24 hours, a software interface was developed by the inventors using the Pipeline Pilot (Accelrys Inc) software platform that allows rapid review of all images that were collected from each primary hybridoma. Further, custom querying tools allow analysis of antibodies that co-localize with ‘marker’ cells related to the immunization (as an example only, transiently tranfected fluorescent protein-fusions of a steroid receptor coactivator). Further, custom querying tools allow for classification of any IF pattern relative to reference marker antibodies or dyes, an important issue when sub-proteomes are used for immunization and/or the cell biology/localization of any immunogen is not well understood; classification by subcellular distribution relative to cell markers immediately provides initial characterization of POI/AOI.

Using a steroid receptor coactivator monoclonal antibody as exemplary demonstration of the invention, the inventors show that this novel use of automated immunofluorescence and analysis is as fast and accurate as needed to make decisions on which primary hybridomas should be expanded, for example. In conjunction with western blotting, immunopreciptitation, siRNA knock down and other biochemical studies, subsequent retesting after expansion, and then single cell cloning, all serve as quality control for the large scale production of monoclonal antibodies specific for the intended immunogen.

In some embodiments of the invention, the inventors also developed and deployed new algorithms, based on the highly dimensional characterization of each mAb signal inherent to automated immunofluorescence screening, that are designed to also recognize additional monoclonal antibody signals that may be related, or unrelated, to the intended purpose of immunization. The mammalian spleen is a robust source of all antibody-producing cells of the animal; however, the cell fusion process is random and inefficient (very few fusions survive to stably produce MAbs). Thus, during the automated immunofluorescence and image-based selection of primary monoclonal antibodies that co-localize with the tagged immunogen (or other cellular marker representing a cytological location where the immunogen is known/expected to be located), the inventors specifically tested the idea that additional immunofluorescent-positive patterns of reactivity would be detected. Therefore, the inventors included positive control antibodies/dyes to ˜10 known but exemplary cytological features for use in a machine-learning approach to search through all IF-positive wells for similar patterns. For example, in some embodiments the inventors used antibodies or dyes to mitochondria, Gogli, lysosomes, the cell surface, endoplasmic reticulum, nuclear lamina, actin, microtubles and endosomes to generate pattern-recognition tools. To this end, while ostensibly searching for steroid receptor coactivator-positive monoclonal antibodies in a working example, the inventors also found monoclonal antibodies that immunofluorescently labeled cells in like-patterns to the positive controls. This process utilized custom software tools, first developed/deployed in Python, and now using Pipeline Pilot. These algorithms applied after image segmentation and feature extraction are based upon, but are not limited to, the application of cross-validated step-wise discrimination, multiple clustering sub-algorithms (K-means, partitioning around medoids, fuzzy analysis, divisive analysis, and agglomerative nesting), and multiple modeling sub-algorithms (support vector machine, linear and nonlinear regression, neural net, and random forest) to group mAbs immunofluorescent patterns by majority rule. The results of the algorithm, which contains both groups related to the POI/AOI and groups unrelated (or “off-target”), are then presented using an web-enabled interface for selection of mAbs for further screening. Numerous monoclonal antibodies were thus selected that produced positive IF patterns that matched the positive controls. One or multiple examples mAbs matching these positive controls were found in the primary hybridoma collection, with select single well examples being chosen for expansion and single cell cloning. Taken together, the process of machine-learning to recognize immunofluorescence-positive patterns to support isolation and expansion of a monoclonal antibody to the intended immunogen, or, interesting monoclonal antibodies to known patterns unrelated to the immunogen both lead to demonstration that the process of high throughput microscopy-based screening of monoclonal antibodies is extremely valuable. Not only are useful monoclonal antibodies obtainable for the immunogen (if made by the mouse or other appropriate animal in sufficient numbers to be successfully fused), but also the mammalian repertoire of antibody-producing clones found in the spleen from any immunonized (or perhaps non-immunized or autoimmune disease model, drug-treated or tumor model or other) mouse is harvestable as a by-product. The latter fact is particularly important when considering the concept of producing monoclonal antibodies to purified or crude factions of proteins or cellular compartments through bulk (shotgun) immunization procedures (see below for examples).

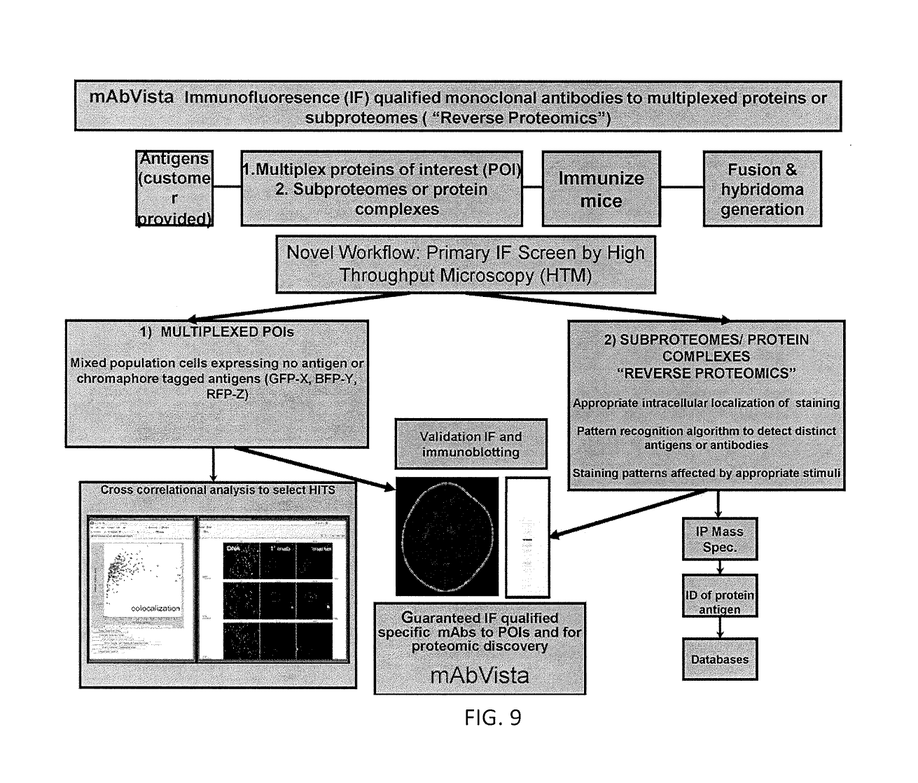

The repertoire of mAbs produced to endogenous or environmental antigens, as described above, provides proof of principle for a broader application of this procedure for producing a range of mAbs to components of subproteomes through multiplex immunization of mice. The procedure involves immunization of mice with subproteomes such as isolated multi-protein complexes or purified or partially purified subcellular compartments, coupled with screening of the hybridomas by high-throughput immunofluorescence imaging and pattern recognition to identify mAbs that detect distinct components or epitopes of the injected subproteome. The positive mAbs can then be used to immunoisolate the corresponding specific native proteins and identify each protein by standard MALDI-TOF or LC-MS/MS mass spectrometry of proteolytic digests of protein bands excised from electrophoresis gels. This “reverse proteomics” approach results in simultaneous identification of protein components and generation of quality specific antibody reagents (see FIG. 9).

The more standard forward discovery proteomics approach involves profiling proteins by mass spectrometry, followed by verification of protein peaks or fractions with specific affinity reagents such as antibodies that are frequently unavailable, or do not have the characteristics required for the specific detection application. The procedure also enables the generation of multiple mAbs from a cell fusion and screening of hybridomas from a single, or only a few, immunized mice, saving substantial time and expense as compared with the conventional procedure of generating mAbs to a single antigen/mouse. The procedure is limited by the immune system of the mouse and potential immuno-dominance of one or a few major proteins of a subproteome. Immuno-dominance can be dealt with by immuno-depletion of major antigens from the subproteome fractions (e.g., depletion of perilipin or adipophilin from lipid droplets) and the range of mAb coverage of components or different epitopes can be expanded by fusions of multiple mice that can be easily handled by high throughput immunofluorescence imaging screening. The range of mAbs reported to be generated by multiplex immunization of mice has been reported to be 40-50 different antibodies (DeMasi, et al., 2005). However, this was done with purified proteins as a mix and screening was done by dot blotting assays with each of the known purified proteins. This approach is not practical for generation of a range of mAbs to unknown components of subproteomes, but it does provide proof-of-principle on the range of mAbs possible to produce from a single set of multiplex immunized mice. Thus the procedure has the potential to generate mAbs to as many as 40-50 protein components of an isolated subproteome. Given the sensitivity comparisons of IF versus ELISA, where IF identified specific, sensitive and versatile antibodies that were missed by ELISA, there may be more than 40-50 potential antibodies per mouse, in certain embodiments.

While antibodies can be produced using this exemplary approach to purified proteins or synthetic peptides or combinations thereof, for example, in some aspects of the invention a very large range of antibodies may be produced using a shotgun approach; however, one task in this embodiment would be in determining which monoclonal antibody clones are useful to keep/expand. As an extension of the reference cellular target antibody or dye colocalization approach, the inventors have an exemplary engineered cell line (PRL-HeLa) that comprises a visible, hormone-regulated transcriptional reporter gene locus, there is partial purification of an exemplary nuclear-factor-enriched cellular fraction for immunization. Hundreds of proteins (or many more, when considering protein variants based upon posttranslational modification) can potentially be immunogens. Through the use of the high-speed immunofluorescence approach and novel image analysis software (Pipeline Pilot), one can screen the primary monoclonal antibodies for IF patterns that specifically localize to a particular visible gene locus. As one can treat the exemplary PRL-HeLa cells with different hormones that have been shown to regulate a particular gene locus (transcriptional agonists or antagonists), one can further survey the primary hybridomas based upon hormone sensitivity. As other natural or engineered subcellular targets that are marked by reference antibody or dye are likely similarly comprised of complex POI, purified or partially-purified sub-proteome immunizations and IF-based screening could yield a wide-range of mAbs (including to posttranslational modifications of POI), in particular embodiments of the invention.

In embodiments of the invention, there is simultaneous production of monoclonal antibodies to components of a subproteome, including at least the following: 1. lipid droplet-associated proteins; 2. subcellular organelle fractions, e.g., Golgi, mitochondria, etc.; 3. cell surface markers associated with stem cell differentiation; 4. biomarkers that differentiate cancer cells expressing wild type androgen receptor (AR) vs. truncated AR (causative to castration-resistant prostate cancer); 5. focal adhesions; 6. caveolae/lipid rafts of plasma membranes; 7. DNA damage nuclear factors; 8. subnuclear splicing islands; 9. multiplex immunogens consisting of peptide conjugates containing posttranslational modification (PTM) sites of interest; 10. affinity isolated protein complexes/machines; 11. drug sensitive and drug resistant cancer cells; and/or 12. exosomes.

In embodiments of the invention, high throughput IF pattern recognition capability is used not only to screen for specific monoclonal antibodies to subproteomes but also to identify those monoclonal antibodies to distinct epitopes and components of subproteomes. These selected monoclonal antibodies in the final step of the process are used to immunoprecipate and identify the specific antigen, for example by mass spectrometry. This process for identification of protein biomarkers has with it built-in affinity reagents for validation.

### IV. Monoclonal Antibodies and Exemplary Methods of Generating

Means for preparing and characterizing antibodies are well known in the art (See, e.g., Antibodies: A Laboratory Manual, Cold Spring Harbor Laboratory, 1988; incorporated herein by reference). Antibodies to HOJ-1 peptides or protein have already been generated using such standard techniques.

The methods for generating monoclonal antibodies (MAbs) generally begin along the same lines as those for preparing polyclonal antibodies. Briefly, a polyclonal antibody is prepared by immunizing an animal with an immunogenic composition in accordance with the present invention and collecting antisera from that immunized animal.

A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The procured blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix or protein A followed by antigen (peptide) affinity column for purification.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified HOJ-1 protein, polypeptide or peptide. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, guinea pig, and hamster is also useful and has been reported. The use of rats may provide certain advantages (Goding, 1986, pp. 60-61), but mice are preferred, with the BALB/c mouse being most preferred as this is most routinely used and generally gives a higher percentage of stable fusions. An exemplary rabbit myeloma is now used to fuse rabbit spleens and make rabbit MAbs; the cell line is termed 240E and is an 8-azaguanine resistant rabbit myeloma (Spieker-Polet et al., 1995, Proc. Natl. Acad. Sci., Vol. 92, pp. 9348-9352).

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. In some embodiments, non-immunized mice, or autoimmune or other disease models are employed.

Following immunization, somatic cells with the potential for producing antibodies, specifically B lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (coding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 S194/5XX0 Bul and Fox-NY; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One exemplary murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid.

The individual cell lines could also be cultured in vitro, where the MAbs are naturally secreted into the culture medium from which they can be readily obtained in high concentrations.

### V. Labels and Labeling

In some embodiments of the invention, an entity is labeled such that its presence and, at least in some cases, location can be determined. In specific embodiments, an antigen is labeled, whereas in certain embodiments an antibody is labeled. The label may be of any kind such that it is visually or otherwise detectable, but in certain embodiments the label is detectable by the nature of having color, being fluorescent, or both.

In some embodiments, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g., ¹²⁵I, ³²P, ¹⁴C, or ³H, or an enzyme, such as alkaline phosphatase, beta-galactosidase or horseradish peroxidase.

A. Fluorescent Labeling

Fluorescent labeling may be accomplished using a chemically reactive derivative of a fluorophore, for example, and common reactive groups include at least isothiocyanate derivatives, such as FITC and TRITC (derivatives of fluorescein and rhodamine); FITC and TRITC are reactive towards primary amines to form a thioureido linkage between the compound of interest and the dye. Succinimidyl esters such as NHS-fluorescein are reactive towards amino groups to form an amido bond. Maleimide activated fluorophores such as fluorescein-5-maleimide react with sulfhydryl groups. The sulfhydryl group adds to the double bond of the maleimide.

Exemplary fluorescent dyes include fluorescein, rhodamine, Alexa Fluors, Dylight fluors, ATTO Dyes, and BODIPY Dyes.

Fluorescent labels generally may be detected via a fluorescence microscope, flow cytometer or some other fluorescence reading instrument.

B. Protein Labeling

Protein labels that are usually covalently attached to a protein of interest to facilitate detection of the labeled protein and/or its binding partners. Labeling strategies can generate covalent attachment of different molecules, including biotin, reporter enzymes, fluorophores and radioactive isotopes, to the target protein. The labeling may occur in vivo or in vitro.

In specific embodiments, an antigen is labeled. The antigen may be labeled as an external attachment to an intact protein or protein fragment, or the antigen may be labeled as the result of the antigen being encoded with a fusion protein whose gene product emits a signal, such as a fluorescent signal, including green fluorescent protein (GFP), enhanced GFP, for example; fusion proteins with a fluorescent entity may be generated by standard molecular biology techniques that are known in the art. Alternatives to GFP include, but are not limited to, in particular, blue fluorescent protein (EBFP, EBFP2, Azurite, mKalama1), cyan fluorescent protein (ECFP, Cerulean, CyPet), and yellow fluorescent protein derivatives (YFP, Citrine, Venus, YPet).

In cases wherein antigen is labeled, the antigen may be labeled by incubating the antigen in a medium containing a radioactive precursor, such as 3H-Thymidine, by iodination or biotinylation of surface proteins, by treatment with radioactive sodium borohydride, or by other published techniques.

### EXAMPLES

The following examples are presented in order to more fully illustrate the preferred embodiments of the invention. They should in no way, however, be construed as limiting the broad scope of the invention.

### Example 1

### A Novel Highthroughput Microscopy-Based Approach to Screen Primary Hybridomas During Production of Monoclonal Antibodies

Monoclonal antibodies (mAb) have been indispensable tools for biomedical research and clinical diagnostics since their discovery in the 1970's. ELISA-based methods have been used for years to rapidly generate antibody-producing primary hybridoma cultures for screening, but the methods provide no insight on whether or not the antibody will be useful in other assays. To overcome this limitation, the inventors implement a high throughput imaging framework to screen primary hybridoma cultures in parallel with ELISA assays. As an example, the inventors use automated imaging approaches to screen for monoclonal antibodies (mAbs) to the amino (N)-terminus of the B isoform of human progesterone receptor (PR-B). Mice were immunized with baculovirus-expressed and purified N-terminus of PRB by standard methods. Following hybridoma fusion, ˜1900 primary cultures were screened by ELISA to define PRA vs. PRB specificity. Simultaneously, primary supernatants were used to immunofluorescently (IF) label a population of PRB-negative MCF-7 cells mixed with MCF-7 stably expressing GFP-PRB growing on optical glass bottom 384 well plates. Automated microfluidic robotics and high throughput microscopy were used to acquire image datasets for all supernatants and a set of control antibodies to subcellular markers. Control images were used to train a classification model that can distinguish between the different organelle patterns which was then applied to the screening dataset to identify hits. Finally, the inventors correlated GFP-PR expression with antibody label intensity to identify PRB-specific hits, and cross-referenced these results with the ELISA data. All imaging and analyses were completed within 20 hours. The inventors provide data demonstrating 1) the ability to define which ELISA-positive hits are also IF-validated, 2) identify PRB-specific antibodies that ELISA missed, and 3) identify several ‘off-target’ antibodies that are potentially useful probes or biomarkers. Thus, the present invention provides a high throughput microscopy approach that facilitates improved efficiency and production of high quality monoclonal antibodies.

The present invention provides an imaging framework to rapidly screen for specific antibodies, including in parallel with ELISA, ensuring high quality and specific hit selection. The present invention provides a framework to identify off-target antibodies that capture specific biomarkers (differentiation markers, organelles, etc.).

The exemplary method employed progesterone receptor (PR) isoforms A and B as an example. The method measured various intensity and texture based features that characterize protein levels and localization from each cell. The inventors used Hotelling T²-test to determine whether or not antibody patterns were statistically different. Pairwise comparisons were made between 18 different monoclonals, and all were determined to be statistically similar (95% confidence interval).

The method also identified “off-target” antibodies that labeled unexpected subcellular compartments that did not co-localize with PR. Off-target Mabs are not against the immunogen and likely reflect antibodies produced by the mouse to environmental antigens. In exemplary screens the inventors automatically identified cytoskeletal and nuclear envelope proteins by obtaining a control dataset for different organelle patterns; training a classifer to recognize these classes; and applying the classifier to identify off-target patterns. Depending on the screen, the classifier identified up to dozens of off-target hits, including intermediate filament, plasma membrane, Golgi apparatus, nuclear envelope/lamina, and nucleoplasms (see FIG. 7). For the present example of PR screen, the inventors manually identified hits in mitochondria, nucleoli, membrane, and nucleus, cytoplasm (see FIG. 7).

It is encompassed in the invention that at least some steps of the methods can be done manually, including without classification models, for example.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of screening primary hybridoma cultures for one or more antibodies of interest, comprising the steps of:
providing a plurality of primary hybridoma cultures generated from immunization of a non-human animal with one or more antigens;
performing a first screen to determine specificity of a test monoclonal antibody for the antigen;
performing a second screen of the test monoclonal antibody by exposing the test monoclonal antibody to a mixture of cells in which some cells in the mixture have the antigen in labeled form and some cells in the mixture lack the antigen;
assaying for in vivo co-localization of the test monoclonal antibody with the antigen; and
assaying for the absence of signal from the label in the cells that lack the antigen.

2. The method of claim 1, wherein the assaying for in vivo co-localization of the test monoclonal antibody with the antigen is further defined as assaying for binding of a labeled second antibody to the test antibody.

3. The method of claim 1, wherein the antigen is a protein, a protein fragment, peptide, cellular extract, an organelle, subcellular structure, subproteome, or mixture thereof.

4. The method of claim 1, wherein one or more of the steps are performed concomitantly.

5. The method of claim 1, wherein the first screen comprises ELISA, western, immunoblot, or a combination thereof.

6. The method of claim 1, wherein the label is fluorescent, colored, or a combination thereof.

7. The method of claim 1, wherein the antigen in labeled form is further defined as being a fusion protein that comprises a protein region that is detectable by color or fluorescence.

8. The method of claim 1, wherein the sensitivity of the test monoclonal antibody is measured by the intensity of the label of the secondary antibody.

9. The method of claim 1, wherein the co-localization has a known co-localization pattern.

10. The method of claim 9, wherein the known co-localization pattern is indicative of a subcellular structure.

11. The method of claim 10, wherein the subcellular structure is an organelle.

12. The method of claim 11, wherein the organelle is selected from the group consisting of nuclei, nucleolus, ribosome, vesicle, rough endoplasmic reticulum, Golgi apparatus, cytoskeleton, smooth endoplasmic reticulum, mitochondria, vacuole, cytosol, lysosome, and/or centriole.

13. The method of claim 1, wherein the co-localization has a known pattern and wherein the method further comprises the step of assaying for antibodies that localize with a subcellular in vivo pattern that is not identical to the known co-localization pattern.

14. The method of claim 1, wherein the method is automated.

15. The method of claim 1, wherein test antibodies from more than one primary hybridoma culture is screened concomitantly.

16. The method of claim 1, wherein the in vivo co-localization is assayed subsequent to treatment of the cells that have the antigen with a cellular signal that results in subcellular movement of the antigen.

17. A method of screening primary hybridoma cultures for one or more antibodies of interest, comprising the steps of:
providing a plurality of primary hybridoma cultures generated from immunization of a non-human animal with one or more antigens;
assaying by secondary immunofluorescence for in vivo localization of monoclonal antibodies from one or more primary hybridoma cultures; and
comparing the localization pattern of the antibodies to the pattern of one or more known cellular features.

18. The method of claim 17, wherein the method is automated.

19. The method of claim 17, wherein the localization of the antibodies is visualized by fluorescence, color, or both.

20. The method of claim 17, wherein a localization pattern of one or more of the monoclonal antibodies is different from the pattern of the one or more known cellular features.

21. A method of producing a plurality of antibodies that recognize a subproteome, comprising the steps of:
providing a plurality of primary hybridoma cultures generated from immunization of a non-human animal with a subproteome;
assaying for in vivo subcellular localization of antibodies from one or more cultures, thereby producing a pattern recognition for the antibodies; and
comparing the pattern to a cellular pattern for one or more known or unknown epitopes or components of the subproteome.

22. The method of claim 21, further comprising using antibodies that recognize the known epitope or component of the subproteome to bind to its respective antigen among a variety of proteins.

23. The method of claim 22, further defined as using the antibodies to recognize the known epitope or component of the subproteome in mass spectrometry, gel electrophoresis, immunoblotting, or a combination thereof.

24. The method of claim 21, wherein the subproteome is a purified or partially purified protein complex.

25. The method of claim 24, wherein the protein complex is a transcriptional regulatory protein complex.

26. A method of screening primary hybridoma cultures for differences in antibody in vivo subcellular localization between two or more cell populations, comprising the steps of:
providing a plurality of primary hybridoma cultures generated from immunization of a non-human animal with a first cell population;
providing a plurality of primary hybridoma cultures generated from immunization of a non-human animal with a second cell population;
exposing the test monoclonal antibody to a mixture of cells in which some cells in the mixture have the antigen in labeled form and some cells in the mixture lack the antigen;
assaying for in vivo subcellular localization of one or more cultures from the respective cell populations.

27. The method of claim 26, wherein the first and second cell populations are further defined as:
a) differentiated vs. undifferentiated cells;
b) cancer vs. non-cancer cells;
c) cancer drug-resistant vs. cancer drug-sensitive cells;
d) hormone or growth factor sensitive and resistant cells;
e) cells from different stages of cancer progression; or
f) cells of different tissue types.
